(19) United States
(12) Patent Application Publication
(10) Pub. No.: US 2009/0177206 A1
(43) Pub. Date: Jul. 9, 2009

(54) INSTRUMENTS, IMPLANTS, AND METHODS FOR FIXATION OF VERTEBRAL COMPRESSION FRACTURES

(75) Inventors: Antony Lozier, Warsaw, IN (US); Nicolas Pacelli, Culver, IN (US); John Dawson, Chaska, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 11/970,581

(22) Filed: Jan. 8, 2008

(51) Int. Cl. A61B 17/58; A61M 29/00

(52) U.S. Cl. 606/93; 606/192; 606/191; 606/92

(57) ABSTRACT

An instrument, and method for using the same, for treating and repairing a vertebral body, which includes a steerable shaft and a distal end portion. The steerable shaft defines a longitudinal axis and includes a proximate portion and a flexible portion along the length. The distal end portion extends from the steerable shaft with an initial diameter, in the collapsed state, and has one or more expanded diameters. After inserting the instrument into the vertebral body, the flexible portion permits the distal end portion of the steerable shaft to be steered away from the longitudinal axis toward a center region within the vertebral body. A cutting surface upon the distal end portion is used to ream cancellous bone within the vertebral body and to create a single void which is off-set form the initial access axis.

### TECHNICAL FIELD

The present invention generally relates to treating a fractured bone. More particularly, the present invention relates to a device and method for treating damage in vertebral bodies.

### BACKGROUND

The human spine consists of a complex set of interrelated anatomic elements including a set of bones called vertebral bodies. Aging and disease, among other conditions, negatively impact the spine. Spinal fractures are a serious concern affecting a wide patient population. One of the largest single segments of this injury category is vertebral compression fractures (VCFs). Osteoporosis, meta-static disease (tumors), and multiple myeloma reduce the structural integrity of the vertebral bodies, predisposing them to fracture. A VCF can result in loss of vertebral height, which in turn can exacerbate neurological conditions or lead to other symptoms. Generally, fractures and loss of height, if not treated, result in a cascade of undesirable injuries. The effects of VCFs can include mild to severe back pain, physical deformity, pulmonary deficit, impaired function, loss of appetite, difficulty sleeping, decreased levels of activity, increased bone loss, and secondary fractures, which all progress toward a significantly reduced quality of life and increased mortality.

VCFs have historically been treated primarily with conservative care including bracing, bed rest, and analgesics. In approximately the last decade, surgical options targeting fixation of the specific fractures have been developed. Surgical options include vertebroplasty and kyphoplasty, both of which include fixation and/or filling of the vertebral body with bone cement. Bone treatment material is often delivered to the treatment site under pressure. Even under controlled conditions and extreme caution, some bone treatment material could enter the blood vessels and venous cavities resulting in the formation of emboli. The flowing blood caries away these emboli and can result in blocked blood vessels in the heart, brain, and other areas. This can result in serious injury, including paralysis and death.

Additionally, existing systems do not provide for void creation that is focused in the center of vertebral bodies, particularly when the axis of the pedicles is largely straight in the anterior-posterior direction. Treatment of VCFs is a multi-faceted challenge, with current devices and methods falling short of addressing all the facets in a satisfactory manner.

Accordingly, there is a continuing need for improved devices and methods for treating damaged vertebral bodies while minimizing risks to the patient.

### SUMMARY

The present invention discloses a device and a method for treating vertebral bodies. One embodiment of the present invention includes a steerable shaft having a length defining a longitudinal axis, a proximate portion, and a flexible portion along the length. A distal end portion extends from the steerable shaft with an initial diameter, which is the collapsed state, and is expandable to one or more diameters along an expansion axis, which is different than the longitudinal axis. The flexible portion permits the distal end portion to be steered away from the longitudinal axis after the distal end portion is inserted into the vertebral body, while the proximal portion is configured for continued alignment with the longitudinal axis. The distal end portion includes a cutting surface for reaming cancellous bone.

In a method according to one embodiment of the present invention, the vertebral body is accessed via the associated pedicle and along an initial access axis. After inserting the instrument of the present invention in its collapsed state, the expandable distal end portion is guided away from the initial access point and toward the center portion of the vertebral bone having cancellous bone material. Next, the distal end portion is mechanically deformed, expanded and rotated, such that at least a portion of the cancellous bone material is removed and a single void, off-set from the initial access axis, is created. A permeable member is inserted into the single void and filled with a bone treatment material.

For those skilled in the art, a more complete understanding of the present invention, and alternative embodiments, will become apparent from the following drawings, their detailed description, and the appended claims. As will be realized, the embodiments may be modified in various aspects without departing from the scope of the invention. Accordingly, the drawings and detailed description are to be regarded as illustrative in nature and not restrictive.

### BRIEF DESCRIPTION OF THE DRAWINGS

FIG. 1A is a cross-sectional view of the superior aspect of a lumbar vertebrae showing cavities formed in the vertebral body and the access paths thereto, in accordance with the prior art;

FIG. 1B is a cross-sectional view of the superior aspect of a lumbar vertebrae showing a cavity formed in the vertebral body and the access path thereto, in accordance with the invention;

FIG. 2 is a front view of an instrument of the invention for forming the cavity of FIG. 1B in the vertebral body, the instrument shown with a steerable shaft aligned along a longitudinal axis and having a distal end portion in a non-expanded state;

FIG. 3 is a partial front view of the instrument of FIG. 2 shown with the distal end portion in an expanded state and off-set from the longitudinal axis by the curved steerable shaft;

FIGS. 4A-4E depict, in isometric view, various embodiments of the expandable distal portion of the instrument of FIG. 2;

FIGS. 5A-5B are front views of another embodiment of an instrument of the invention for forming the cavity of FIG. 1B, with the distal end portion in the non-expanded state and the expanded state, respectively;

FIGS. 6A and 6B are side views of embodiments of an expanded distal end portion having side expansion control wires and a central expansion control wire, respectively, and side steering control wires;

FIG. 7A is a cross-sectional view of the superior aspect of a lumbar vertebrae showing an initial access path formed in accordance with a method of the invention;

FIG. 7B is a cross-sectional view showing insertion of the instrument of FIG. 2 through the initial access path of FIG. 7A and toward a central portion of the cancellous bone region of the vertebral body;

FIG. 7C is a cross-sectional view showing a first incremental expansion of the distal end portion for reaming a cavity in the cancellous bone region;

FIG. 7D is a cross-sectional view showing a second incremental expansion of the distal end portion for reaming an incrementally larger cavity in the cancellous bone region;

FIG. 7E is a cross-sectional view showing the reamed-out cavity of desired size;

FIG. 8 is a schematic view of one embodiment of a permeable member for inserting into the cavity depicted in FIG. 7E.

FIGS. 9A and 9B are schematic views of another embodiment of a permeable member for inserting into the cavity depicted in FIG. 7E, shown in an insertion state and a filled state, respectively.

FIGS. 10A and 10B are schematic views of yet other embodiments of a permeable member for inserting into the cavity depicted in FIG. 7E, shown with reinforcing elements;

FIG. 11A is a cross-sectional view showing insertion of the permeable member of FIG. 9A into the reamed-out cavity of FIG. 7E; and

FIG. 11B is a cross-sectional view showing the permeable member of FIG. 9B filled to substantially conform to the shape of the reamed-out cavity of FIG. 7E.

### DETAILED DESCRIPTION

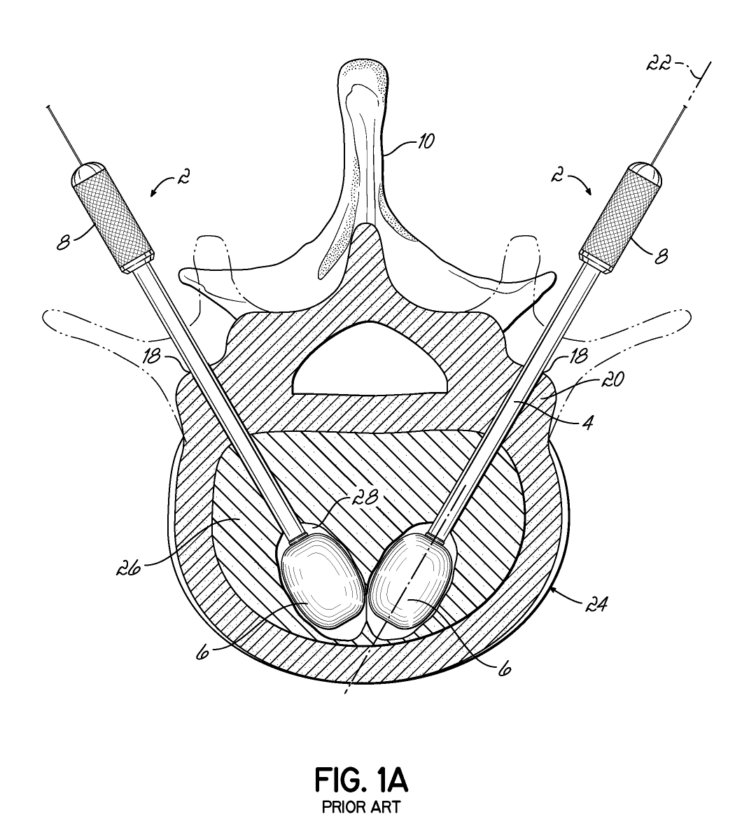

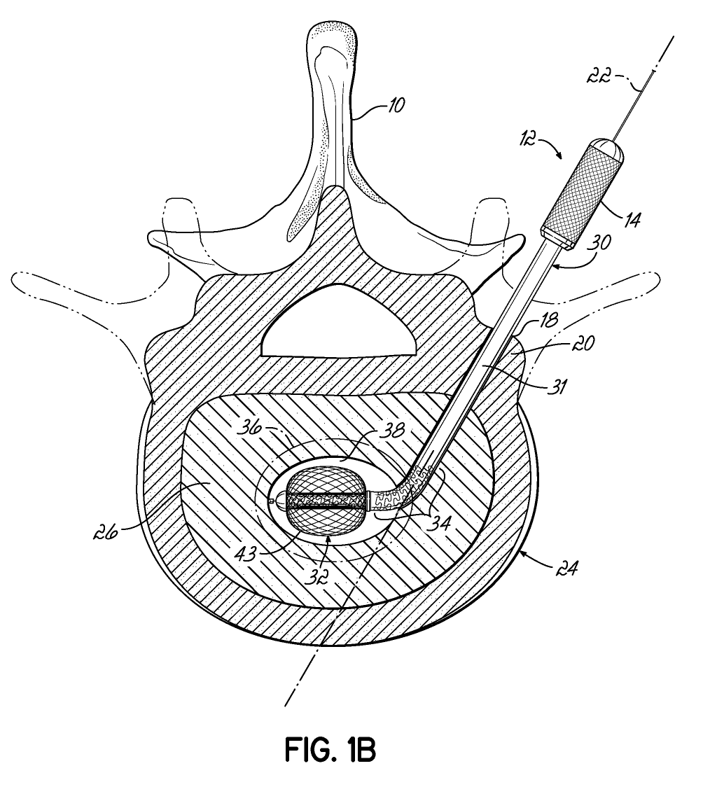

One prior surgical instrument and method commonly used for vertebral body repair, referred to as kyphoplasty, is illustrated in FIG. 1A from the viewpoint of a cross-section of the superior aspect of a lumbar vertebra 10. The instrument 2 includes a rigid shaft 4, wherein the inserted end includes a balloon tamp 6 and the opposing end a handle 8. Procedurally, the surgery begins with insertion of the shaft 4 and balloon tamp 6 through one of two pedicle access points 18 of the associated pedicles 20 of vertebra 10 and along an initial longitudinal axis 22 into the vertebral body 24. Once the balloon tamp 6 of the instrument 2 is within the cancellous bone region 26 of the vertebral body 24, the surgeon expands the balloon tamp 6 to create a cavity 28 by compression of the surrounding cancellous bone. This procedure is repeated through a pedicle access point 18 in the other of the associated pedicles 20 to create another cavity 28, as shown. After withdrawal of the instrument 2 and balloon tamp 6, the surgeon fills the cavities 28 with a viscous bone filler material (not shown) that will provide structural aid to the vertebra 10. Bone filler material is commercially available and may include, for example, polymethylmethacrylate (PMMA), bisphenol-A-glycidyldimethacrylate (BIS-GMA) materials such as CORTOSS™, dental composites, gypsum-based composites, polyurethane, etc.

Contrast the prior surgical instrument 2 now with FIG. 1B diagramming, most generally, one embodiment of an instrument 12 having the capability of deviating from the axis of insertion (e.g., longitudinal axis 22) into the central portion of vertebral body 24 to form a single cavity, or void, rather than requiring formation of two cavities (e.g., cavities 28). To that end, the instrument 12 comprises a handle 14, a steerable shaft 30, and a distal end portion 32 to be inserted into the vertebra 10. The steerable shaft 30 includes a flexible portion 34, which enables the distal end portion 32 of the instrument 12 to be steered away from the longitudinal axis 22 toward a central portion 36 of the vertebral body 34, and a proximal portion 31 adapted for continued alignment with the longitudinal axis 22. The term “steered” as used herein contemplates both active and passive steering, e.g., actively manipulating the shaft 30 to change its direction or passively guiding the shaft 30 over a curved instrument (such as a guide wire) defining a desired pathway, wherein the flexible portion 35 enables the active or passive steering. Once the instrument is within the central portion 36, the distal end portion 32, having cutting surfaces, is used for reaming cancellous bone to create a central and single void 38. The distal end portion 32 can also have a cutting tip (not shown) for facilitating entry and “drilling” over a guide wire.

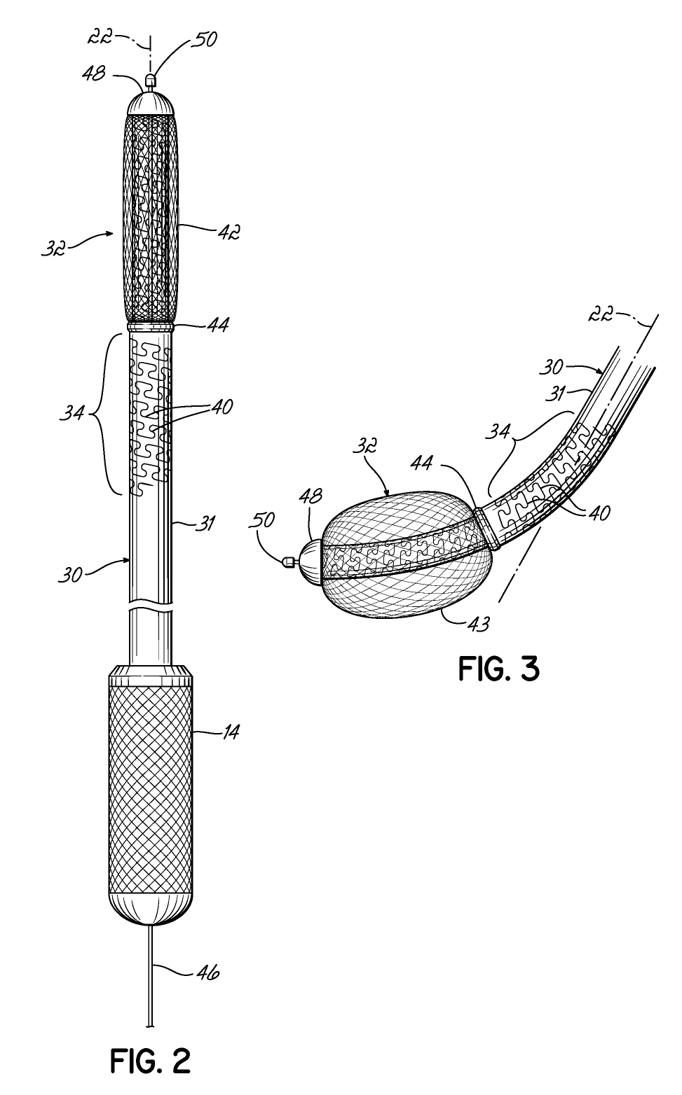

FIGS. 2 and 3 depict a partial front view of the instrument 12 of the present invention in greater detail. FIG. 2 illustrates the instrument 12 in a non-expanded and non-modified directional state; that is, the distal end portion 32, the steerable shaft 30, and handle 14 lie along a single longitudinal axis 22. As shown, and by way of example only, the steerable shaft 30 may include a plurality of laser cuts 40 within the flexible portion 34, which allows distal end portion 32 of the instrument 12 to be steered away from the longitudinal axis 22, as shown in FIG. 3, while the proximal portion 31 remains aligned along the longitudinal axis 22. In an alternate embodiment, the steerable shaft 30 is a rod, tube, or wire of an elastic but tough material, (e.g., certain polymers or elastomers, or superelastic metal alloys).

Referring again to FIG. 2, another feature of the present embodiment is an expandable reaming device 42 on the distal end portion 32. The expandable reaming device 42 extends between a proximal end 44 and a distal tip 48. The expandable reaming device 42 may comprise a wire structure surrounding a guide wire 46, where the guide wire 46 extends proximally beyond the handle 14 and distally beyond the distal tip 48 to a guide wire tip 50. The guide wire can also be used without a tip. The end of the guide wire 46 can be shaped such that it cuts its own way into the bone, such as bevel or trocar tip. The guide wire 46 may be constructed of stainless steel, a nickel-titanium alloy, or other suitable material. The steerable shaft 30 may extend into the distal end portion 32 within the expandable reaming device 42, as shown, or it may terminate at the proximal end 44 of distal end portion 32.

In one particular embodiment of utilizing the instrument 12 (discussed in greater detail below), a previously inserted guide wire 46 may be used for guiding the distal end portion 32 of the instrument 12 into the vertebral body 24. In another embodiment, the guide wire 46 includes a pre-curved distal portion to facilitate steering the distal end portion 32 of the instrument 12 into the vertebral body 24. Yet another use of the guide wire tip 50 and/or the distal tip 48 (collectively and generically referred to as the tip portion) is to act as a stop member to engage the distal end portion 32 and thereby preventing the distal end portion 32 from moving past the tip portion. An alternate wire construction eliminates the “stop” feature and the instrument 12 is allowed to pass over the end, with directional guidance from the guide wire 46.

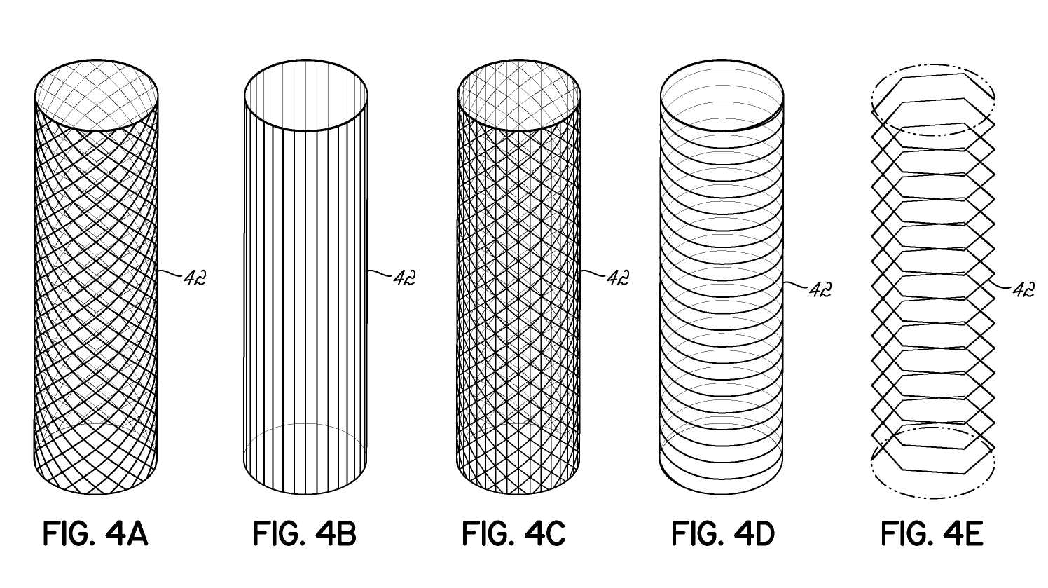

The expandable reaming device 42 may have any one of several structures, particularly a structure and material having a preferential ability to cutting cancellous bone over cutting cortical bone tissues. For example, five wire structures that would be suitable for the present invention are depicted in isometric view in FIGS. 4A-4E. FIG. 4A depicts a braided wire structure also shown on the instrument 12 of FIGS. 2 and 3. FIG. 4B depicts a straight wire structure, and FIG. 4C depicts a composite or hybrid structure combining the braided and straight wire structures of FIGS. 4A and 4B. FIGS. 4D-E, respectively, depict helical and zig-zag wire structures. It may be appreciated that additional hybrid structures may be formed by combinations of the wire structures depicted in FIGS. 4A, 4B, 4D and 4E. It may be further appreciated that expandable reaming device 42 is not limited to the exemplary wire structures shown and described.

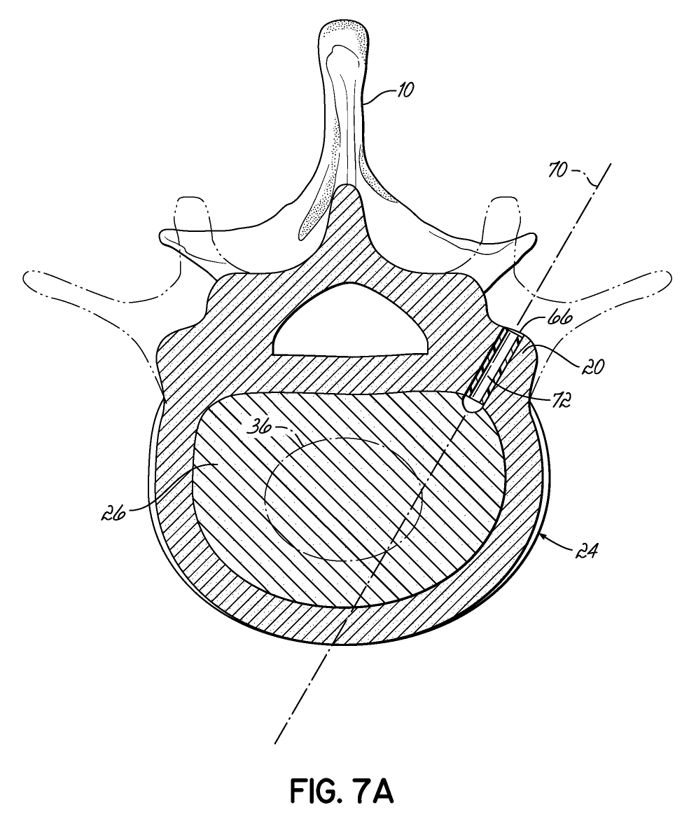

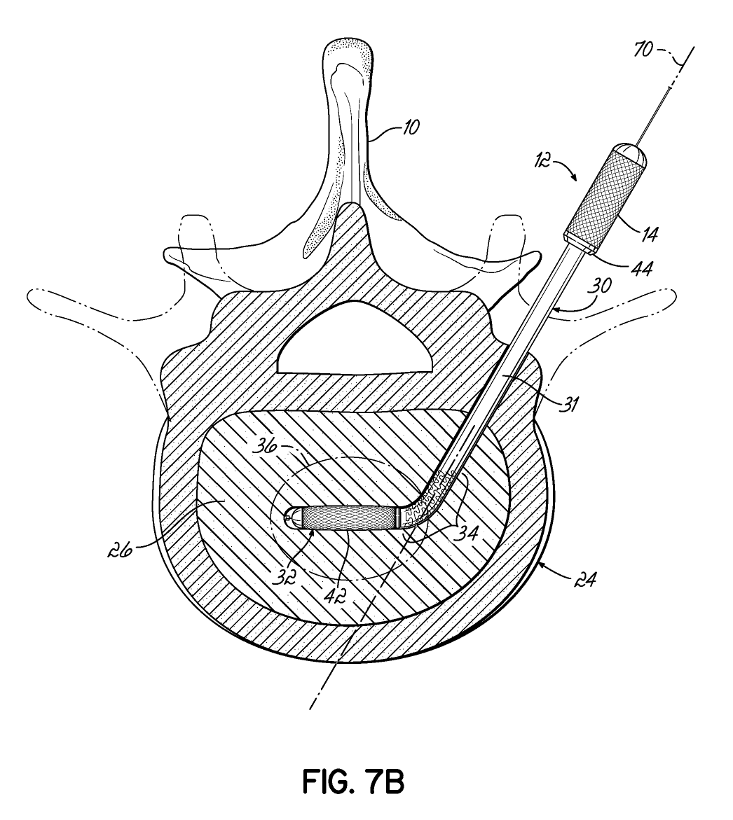

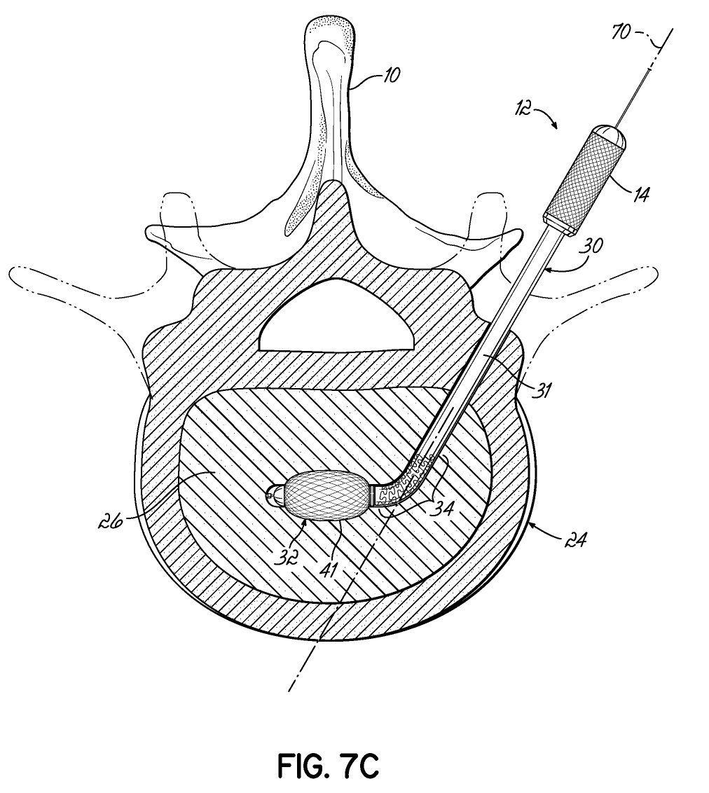

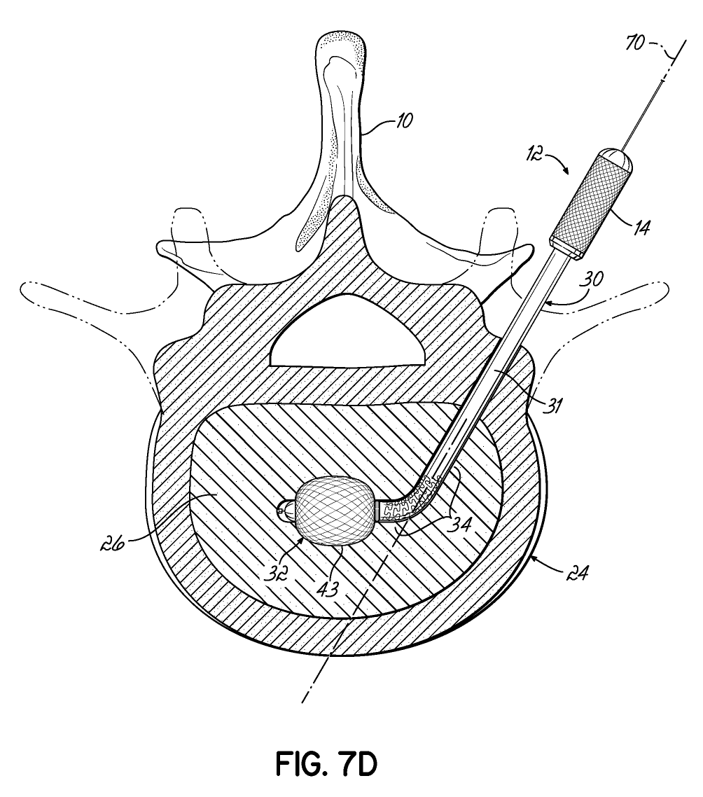

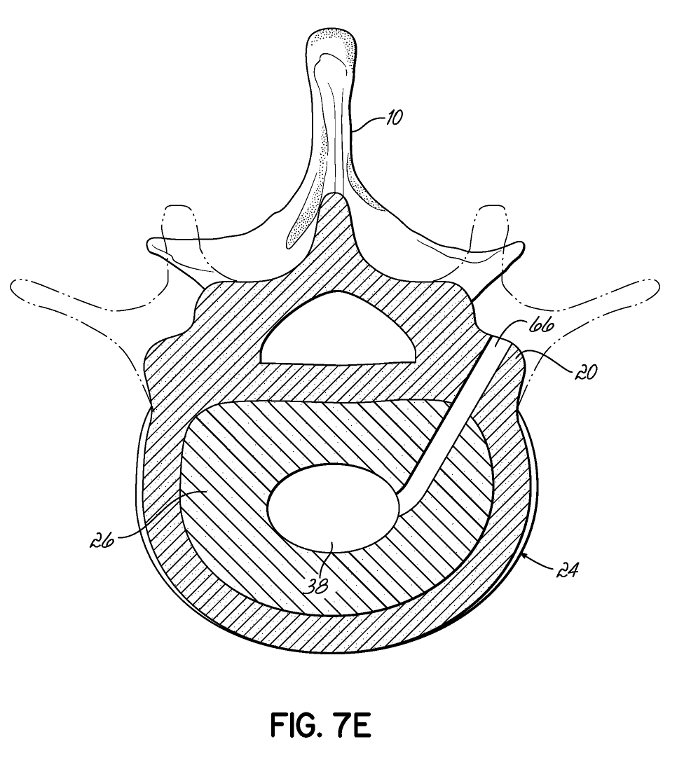

Referring again to FIGS. 2 and 3, one embodiment for expansion control is generally described. The instrument 12 may include one or more expansion control wires (not shown but described in greater detail below) connected to the distal tip 48 and extending along the steerable shaft 30 toward the handle 14. The proximal end 44 of distal end portion 32 is fixed to shaft 30. The expandable reaming device 42 is manipulated using the expansion control wires to pull the distal tip 48 toward the proximal end 44 fixed to the steerable shaft 30 to achieve a fully expanded reaming device 43 (or a partially expanded reaming device 42 discussed hereafter in FIG. 7C). The expansion control wires can alternately be connected to a more proximal point along the shaft 30 in the distal end portion 32 and still effect guidance on the distal tip 48. This manipulation allows the expandable reaming device 42 to expand from an initial diameter in a direction along an expansion axis different than the longitudinal axis 22, such as transverse to the longitudinal axis 22, to an expanded diameter greater than the initial diameter.

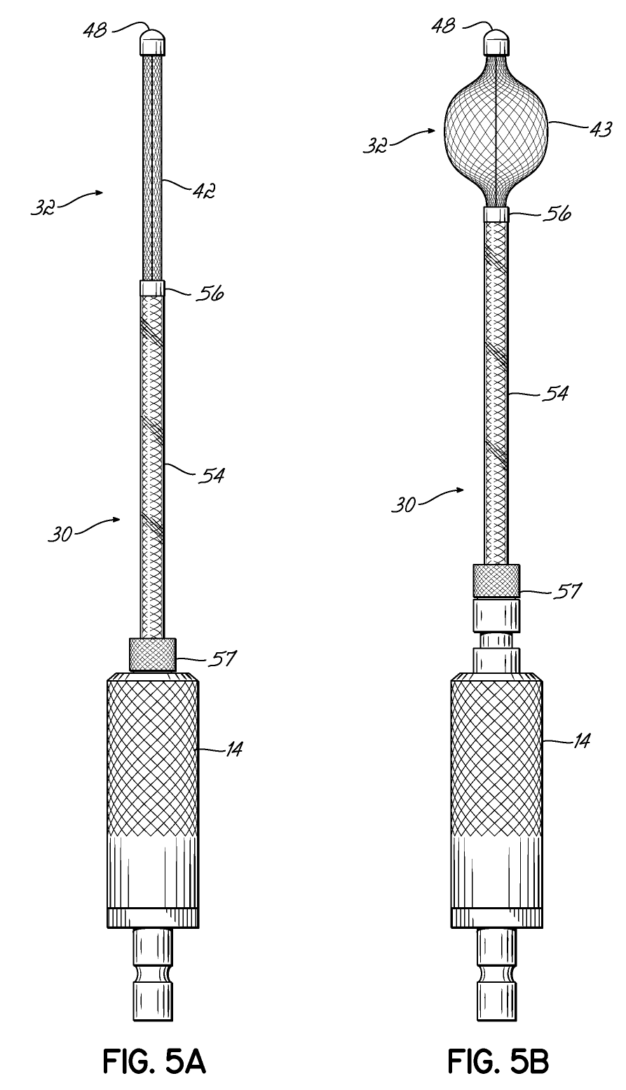

Another means of expansion control is shown in FIGS. 5A and 5B. Beginning with FIG. 5A, a braided flexible wire forms both the expandable reaming device 42 of the distal end portion 32 and the flexible portion 34 of the steerable shaft 30. As shown, the braided flexible wire forms the entire shaft 30, but this is not necessary. An actuating nut 57 is provided at the junction of handle 14 and steerable shaft 30. Also shown is a flexible, non-expandable sheath 54 surrounding the braided flexible wire along steerable shaft 30 and a rigid annular member 56 defining the boundary between the steerable shaft 30 and the distal end portion 32 (i.e., defining the proximal end 44). To expand the expandable reaming device 42, the actuating nut 57 is advanced toward the distal tip 48, which also advances the sheath 54 and rigid annular member 56. As shown in FIG. 5B, the braided flexible wire expands in the distal end portion 32 to form the expanded reaming device 43 while the sheath 54 and rigid annular member 56 act to restrain the braided flexible wire from expanding along the shaft 30. In addition, by using the rigid annular member 56 and the flexible non-expandable sheath 54, the surgeon may fully extend the reaming device 42 without loosing the flexible or steerable nature of the shaft 30 of the instrument 12.

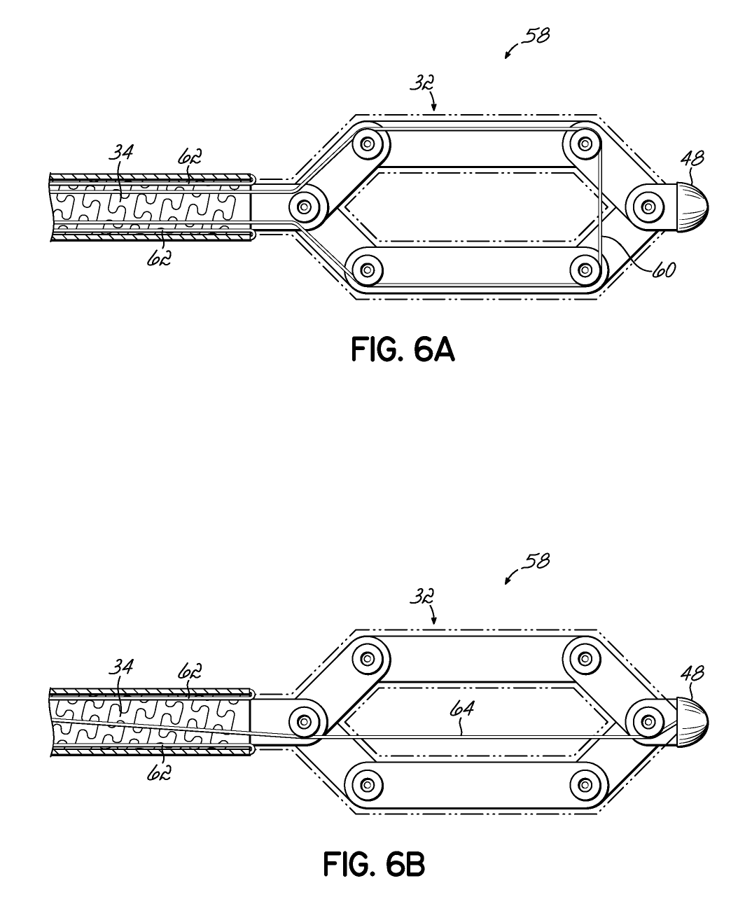

FIGS. 6A and 6B depict other embodiments of the expandable reaming device 42 in conjunction with expansion control wires and steering wires. Rather than a braided wire, FIGS. 6A-6B illustrate an expandable jack device 58. FIG. 6A also shows a side expansion control wire 60 starting at or adjacent to the handle 14 (not shown) that extends around the perimeter of the expandable jack device 58 and continues back along the shaft 30 toward the handle 14. In another embodiment of controlled expansion of the expandable jack device 58, as shown in FIG. 6B, a central expansion control wire 64 is fixed at the distal tip 48 and extends toward the handle 14 (not shown). In either FIG. 6A or 6B, the surgeon pulls corresponding wires (or uses some mechanism that controls the wires, such as a knob, dial, switch, trigger, etc.) to initiate expansion of distal end portion 32 of the instrument 12 for reaming.

Also shown in FIGS. 6A and 6B is one embodiment for steering wires 62. For this particular embodiment, there is a side steering wire 62 for opposing sides of the flexible portion of the steerable shaft 34. These steering wires 62 are fixed within the distal end portion 32 (for illustration only, shown at the boundary between the flexible portion of the steering shaft 34 and the expandable jack device 58 or other expandable reaming device) and extend toward the handle 14 (not shown). Upon manipulation of one or more of the steering wires 62, the surgeon is able to steer the distal end portion 32 away from the longitudinal axis 22.

The previously described embodiments of the structural elements of the instrument 12 of the present invention, and other obvious modifications, may be used in a manner for treating a vertebral body 24 through its associated pedicle 20 and in preparation of other procedures, such as vertebroplasty. Advantageously, the instrument 12 of the present invention is used in a manner to create a single void 38 off-set from an initial access axis of a single pedicle access point and toward a center portion 36 of the cancellous bone region 26 of the vertebral body 24.

FIGS. 7A-7E illustrate one exemplary method of creating the off-set single void 38 within the cancellous bone region 26. The procedure begins in FIG. 7A with showing an initial access point 66 through an associated pedicle 20 to define an initial access axis 70. The initial access may be in a posterior-lateral direction, as shown, or other suitable direction as appropriate. After scalpel incision, a Jamshidi stylet, having an associated tube 72 with shaft (not shown) through the center and a protruding trocar tip (not shown), is inserted at the single access point 66 and pushed toward the cancellous bone region 26 along initial access axis 70. Upon reaching the cancellous bone region 26, the shaft of the Jamshidi stylet is removed leaving the tube 72 in place.

Referring to FIG. 7B, a stylet (not shown) with a pre-curved guide wire 46 is inserted through the tube 72 and further into the cancellous region 26 along a desired pathway. Once the desired location is reached (described in greater detail below), with the guide wire 46 extending past the stylet to the desired position, the stylet is removed and the instrument 12 of the present invention is inserted over the guide wire 46. Alternately, the stylet and guide wire 46 may form a single instrument, connected at least at the tip, such that the stylet is not removed from the guide wire when the tip reaches the desired position. In another embodiment, the instrument 12 is inserted without the aid of the guide wire and manually steered into the desired location by using a steering means, such as the steering wires 62 described previously. In yet another embodiment, a pre-curved or curveable guide tube is inserted first and followed by inserting the instrument 12 through and beyond the guide tube. Other means of inserting a pedicle device would be known within the art.

The desired final location of the distal end portion 32 of the instrument 12 is off-set from the initial access axis 70 toward a central portion 36 of the cancellous bone region 26 within the vertebral body 24. This destination is reached by manipulating the flexible portion 34 of steerable shaft 30 such that the distal end portion 32 moves away from the initial access axis 70 along the desired pathway. The proximal portion 31 of steerable shaft 30 remains aligned along the initial access axis 70. As with the meaning of the term “steerable,” the term “manipulating” as used herein encompasses both active and passive manipulation.

After reaching this desired location, the surgeon begins the process of creating a single void 38 by expanding and rotating the expandable reaming device 42, either continuously, or incrementally as shown in FIGS. 7C and 7D. FIG. 7C depicts a partially expanded reaming device 41 of intermediate diameter for forming an incremental portion of the single void 38, while FIG. 7D depicts the fully expanded reaming device 43 of final diameter for forming the final single void 38 of desired dimension. The reaming procedure includes expansion as well as rotation or other manipulation of the distal end portion such that at least a portion of the cancellous bone region is removed, and advantageously such that cortical bone is not removed. In some situations, it may be appropriate to incrementally and progressively expand and rotate the distal end portion 32 in two (as shown) or more incremental steps. In other words, the diameter of the distal end portion 32 increases, in stepped or incremental fashion, from an initial unexpanded diameter to one or more intermediate partially expanded diameters, and then to a final expanded diameter, with rotation at each expanded diameter. Alternatively, the steps may occur simultaneously or continuously. Still other situations may require a single expansion to a diameter equal to the desired diameter of the single void prior to or without a rotation. The precise manner by which the cancellous bone tissue is removed will be according to the preference of the surgeon for a particular patient and may include other steps not included here.

Removal of the instrument yields a single void 38 connected with a path accessible from the single access point 66 of the associated pedicle 20 as illustrated in FIG. 7E.

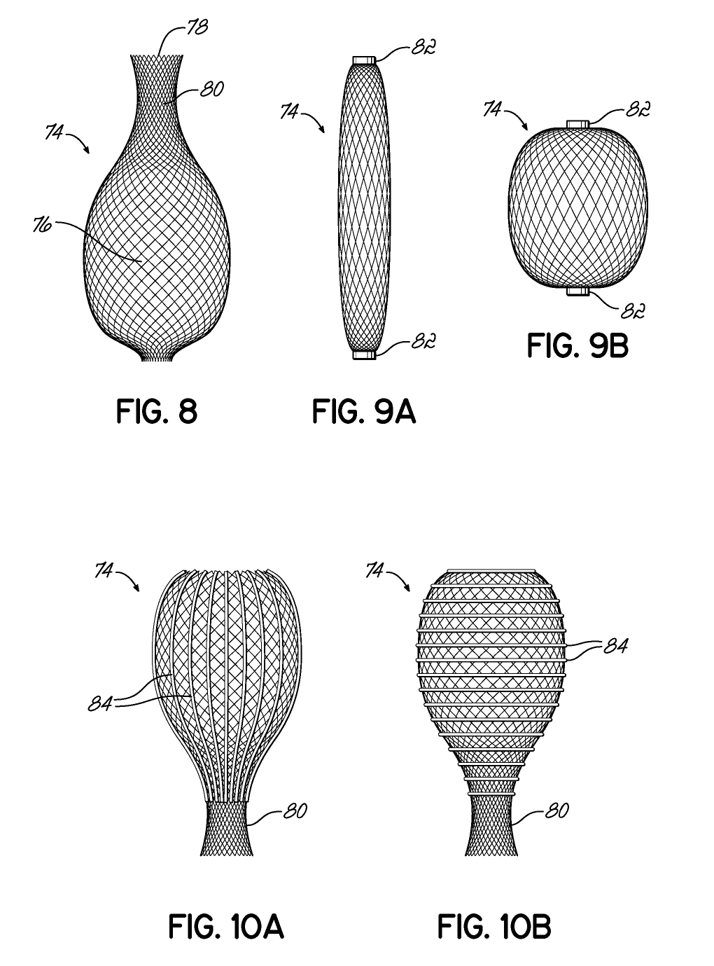

The vertebra is now prepared to receive a permeable member, which is adapted to receive and substantially contain a bone treatment material. This permeable member may be one of several different shapes and of various materials. FIGS. 8 through 10B depict four such permeable members.

FIG. 8 shows one permeable member 74 having an essentially circular-hollow barrel shape 76 with an interior cavity, a port 78, a neck 80, and is generally of an essentially circular-hollow barrel shape. The permeable member may be constructed of various materials, including woven, braided, knit, felted, or electrospun materials, or other materials that are capable of being manipulated (compression and expansion) while containing a bone treatment material by at least partially resisting leaks of said bone treatment material.

Another shape for the permeable member 74 is an elliptical-hollow shape such as in FIG. 9A, which is expandable into an essentially spherical shape upon being filled with the bone treatment material, as shown in FIG. 9B. FIGS. 9A and 9B also demonstrate a restrictive band 82, which aids in containing the bone treatment material.

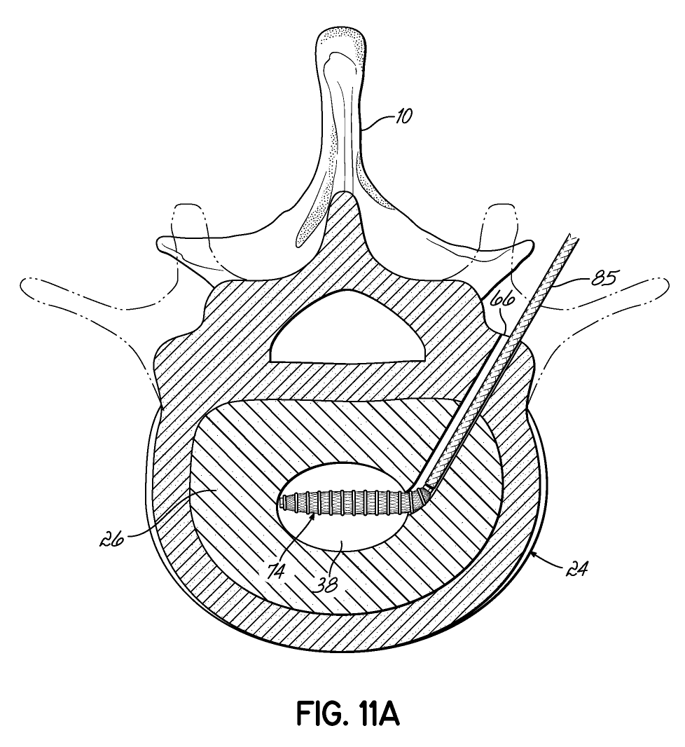

In particular situations, it may be necessary or desirable to provide structural reinforcement to the permeable member. Thus, the permeable member 74 may also include one or more reinforcing filaments 84 similar to those shown in FIGS. 10A and 10B. Construction of these reinforcing filaments 84 is typically of a material different from the permeable member 74, including a suitable superelastic, shape-memory, or elastic or spring-like metallic material such as NiTi, 17-4 PH™ stainless steel, or Ti-6Al-4V. Various polymers could also be used for the reinforcing filaments 84, particularly where the polymer is stiffer than the material of the permeable member 74. Another particularly useful feature of the reinforcing filaments is the creation of shape memory in the permeable member 74. After creating the shape memory, the permeable member 74 is collapsed for entry into the vertebra 10 (described further below) and allowed to expand to the shape in memory (which may then be expanded to substantially conform to the shape of the single void 38). Otherwise, the permeable member 74 will freely expand with the insertion of the bone treatment material to approximately the shape of the single void 38. A final method would be to collapse the permeable member 74 for insertion and then allow expansion to fill the shape of the single void 38 prior to the introduction of the bone treatment material.

Another feature of the permeable member 74 is the inclusion of a material that is applied in a desired pattern to portions of the permeable member 74 and affects the permeability to the bone treatment material. The controlled permeability enables a controlled amount of bone treatment material to permeate through the permeable member 74 to penetrate voids and fissures in the vertebral body 24.

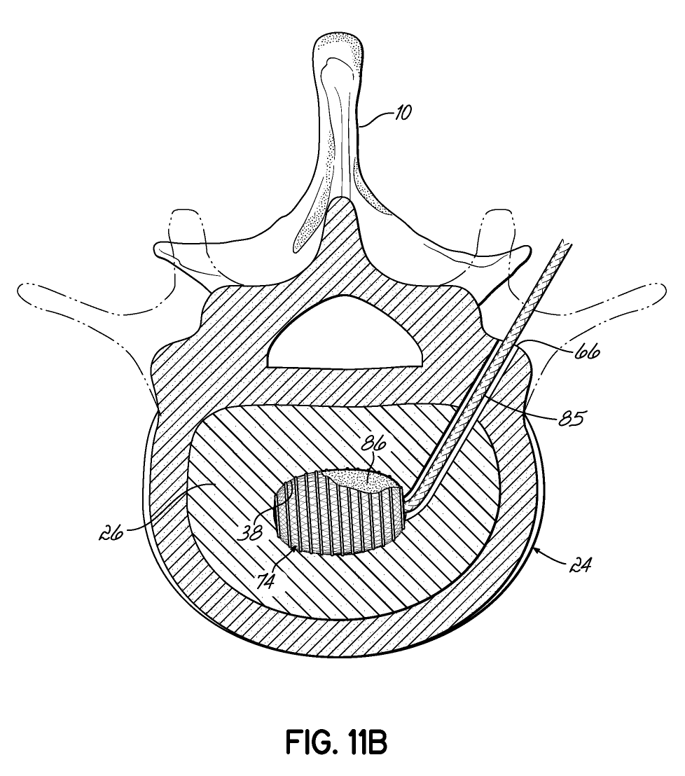

Turning now to FIG. 11A to complete the method of treating a vertebral body, a permeable member 74 is attached to an insertion tool 85 and inserted into the single void 38 via the single access point 66. Any permeable member described previously or having other appropriate structure may be used. Upon proper placment of the permeable member 74, a bone treatment material 86 is introduced into and fills the permeable member 74. Expansion of the permeable member 74 may occur in any manner, including those detailed above. FIG. 11B illustrates the permeable member 74 filled to substantially conform to the shape of the reamed out single void 38 and prior to the removal of the insertion tool 85. After removal of the insertion tool, the permeable member 74 remains within the single void 38 to aid in restoring the height of a collapsed vetrebral body.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. For example, in addition to the off-axis steering discussed in detail above, the instrument of the present invention will also be useful in linear access configurations, where the distal end need not be off-set from the initial access axis. The invention in its broader aspects is therefore not limited to the specific details, representative apparatus and method and illustrative examples shown and described. Accordingly, departures may be made from such details without departing from the scope of the general inventive concept.

What is claimed is:

1. An instrument for treating a vertebral body comprising:
a steerable shaft having a length defining a longitudinal axis, a proximal portion, and a flexible portion along the length;
a distal end portion extending from the steerable shaft having an initial diameter in a collapsed state and being expandable along an expansion axis different than the longitudinal axis to one or more expanded diameters greater than the initial diameter,
wherein the flexible portion is configured to permit the distal end portion to be steered away from the longitudinal axis following entry of the distal end portion into the vertebral body with the proximal portion configured for continued alignment with the longitudinal axis, and
wherein the distal end portion includes cutting surfaces for reaming cancellous bone.

2. The instrument of claim 1 wherein the distal end portion is flexible and configured to further permit the distal end portion to be steered away from the longitudinal axis.

3. The instrument of claim 1 wherein the cutting surfaces are configured to preferentially cut cancellous bone without cutting cortical bone.

4. The instrument of claim 1 further comprising one or more expansion control wires connected to the distal end portion and extending along the shaft, wherein upon manipulation, the one or more expansion control wires are configured to pull a distal end of the distal end portion toward the shaft to expand the distal end portion transverse to the longitudinal axis.

5. The instrument of claim 1 further comprising one or more steering wires connected to the distal end portion and extending along the shaft, wherein upon manipulation, the one or more steering wires are configured to steer the distal end portion away from the longitudinal axis.

6. The instrument of claim 1 further comprising a guide wire extending through the shaft and distal end portion and having a tip portion extending distally beyond the distal end portion for guiding the distal end portion into the vertebral body.

7. The instrument of claim 6 wherein the guide wire includes a pre-curved distal portion to facilitate steering the distal end portion away from the longitudinal axis following entry into the vertebral body.

8. The instrument of claim 6 wherein the tip portion is configured to act as a stop member to engage the distal end portion and thereby prevent the distal end portion from moving past the tip portion.

9. The instrument of claim 1 wherein the flexible portion comprises a plurality of laser cuts in the steerable shaft.

10. The instrument of claim 1 wherein the flexible portion comprises a braided flexible wire and a flexible, non-expandable sheath surrounding the braided flexible wire.

11. The instrument of claim 1 wherein the steerable shaft and the distal end portion comprise a flexible, expandable wire, the instrument further comprising a rigid annular member around the flexible, expandable wire defining a boundary between the steerable shaft and the distal end portion, and a flexible, non-expandable sheath surrounding the steerable shaft, wherein the rigid annular member and the flexible, non-expandable sheath cooperate to constrain expansion of the flexible, expandable wire along the length of the steerable shaft while maintaining the flexible portion for steering and permitting expansion of the distal end portion.

12. A method of treating a vertebral body through its associated pedicle, said method comprising:
accessing the vertebral body through a single access point in the associated pedicle and along an initial access axis;
introducing an expandable reaming device through the single access point and to a cancellous bone region of the vertebral body, the expandable reaming device configured linearly along the initial access axis and having an expandable distal end portion, a proximal portion, and a flexible shaft, with the expandable distal end portion in a collapsed state;
modifying the expandable reaming device with the expandable distal end portion moving away from the initial access axis toward a center portion of the cancellous bone region and the proximal portion remaining along the initial access axis;
expanding the expandable distal end portion by mechanical deformation and rotating the expandable distal end portion to remove at least a portion of the cancellous bone region to form a single void off-set from the initial access axis and in the center portion;
inserting into the single void a permeable member adapted to receive and substantially contain a bone treatment material therein; and
introducing the bone treatment material into the permeable member to fill the permeable member,
wherein the filled permeable member substantially conforms to the shape of the single void.

13. The method of claim 12 further comprising:
inducing a controlled amount of the bone treatment material to permeate through the permeable member to penetrate voids and fissures in the vertebral body.

14. The method of claim 12 wherein the permeable member includes a material applied in a desired pattern to portions thereof whereby the material affects the permeability of the member.

15. The method of claim 12 wherein the step of accessing is in a posterior-lateral direction.

16. The method of claim 12 wherein the steps of expanding and rotating the expandable distal end portion occur progressively, including expanding to a first expanded diameter and rotating, followed by expanding to a next incremental expanded diameter greater than the first expanded diameter and rotating, and repeating the expanding and rotating until the next incremental expanded diameter is equal to a desired diameter of the single void.

17. The method of claim 12 wherein the steps of expanding and rotating the expandable distal end portion occur simultaneously.

18. The method of claim 12 wherein the steps of expanding and rotating the expandable distal end portion occur in sequence, wherein the distal end portion is first expanded to a diameter equal to the desired diameter of the single void, followed by rotation to remove a portion of the cancellous bone region to form the single void of the desired diameter.

19. The method of claim 12 wherein the permeable member is expandable, and wherein the step of introducing the bone treatment material expands the permeable member to the shape of the single void.

20. The method of claim 12 wherein the permeable member is expandable, the method further including expanding the permeable member to the shape of the single void prior to the step of introducing the bone treatment material.
